(19) United States
(12) Patent Application Publication
(10) Pub. No.: US 2022/0133184 A1
(43) Pub. Date: May 5, 2022

(54) LOW-PROFILE WEARABLE MEDICAL DEVICE

(71) Applicant: MEDTRONIC MINIMED, INC., Northridge, CA (US)

(72) Inventors: Ellis Garai, Studio City, CA (US); Al L. McLevish, Roseville, MN (US); Jesse Hefner, Minneapolis, MN (US); David Yueh-Hua Choy, San Gabriel, CA (US)

(21) Appl. No.: 17/086,178

(22) Filed: Oct. 30, 2020

(51) Int. Cl. A61B 5/145; A61B 5/00

(52) U.S. Cl. CPC A61B 5/14532; A61B 5/6832; A61B 2560/0214; A61B 2562/166; A61B 2562/164; A61B 5/0024

(57) ABSTRACT

Wearable medical devices are provided. An exemplary wearable medical device includes a flexible printed circuit board assembly (PCBA), a battery cell having a first terminal and a second terminal, a sensor, and a uniaxially electrically conductive adhesive electrically connected to the flexible PCBA, to the first terminal and the second terminal of the battery cell, and to the sensor. An exemplary sensor is a glucose sensor

### TECHNICAL FIELD

The present technology is generally related to wearable medical devices, and more particularly to low-profile wearable medical devices having reduced height and/or footprint area.

### BACKGROUND

The use of wearable medical devices, such as continuous glucose monitor (CGM) devices, is increasing. Wearable medical devices may provide biometric monitoring and reporting relating to the health of a wearer. In many health monitoring applications, a wireless sensor in the wearable medical device is attached directly to or under the user's skin to measure certain data. This measured data can then be utilized for a variety of health-related applications.

Wearable medical devices allow for continuous monitoring of a user's health. However, due to the continuous nature of the monitoring, users are particularly concerned about comfort and the possible obtrusiveness of these devices in certain situations.

Accordingly, it is desirable to provide a low-profile wearable medical device with a reduced height. Also, it may be desirable to provide a wearable medical device with a smaller footprint. In addition, it is desirable to provide a wearable medical device having fewer components. Furthermore, other desirable features and characteristics will become apparent from the subsequent detailed description and the appended claims, taken in conjunction with the accompanying drawings and the foregoing technical field and background.

### BRIEF SUMMARY

The subject matter of this disclosure generally relates to wearable medical devices. An exemplary wearable medical device includes a flexible printed circuit board assembly (PCBA), a power source, and a uniaxially electrically conductive adhesive electrically connecting the flexible PCBA and the power source.

Further, in an exemplary embodiment, the power source has a first end and an opposite second end, the flexible PCBA has a distal portion located adjacent to and electrically connected to the first end of the power source, and the flexible PCBA has a proximal portion located adjacent to and electrically connected to the second end of the power source. In an exemplary embodiment, a first portion of the uniaxially electrically conductive adhesive electrically connects the distal portion of the flexible PCBA and the first end of the power source, a second portion of the uniaxially electrically conductive adhesive electrically connects the proximal portion of the flexible PCBA and the second end of the power source, and the first portion of the uniaxially electrically conductive adhesive and the second portion of the uniaxially electrically conductive adhesive are discontinuous. In an exemplary embodiment, a first portion of the uniaxially electrically conductive adhesive electrically connects the distal portion of the flexible PCBA and the first end of the power source, a second portion of the uniaxially electrically conductive adhesive electrically connects the proximal portion of the flexible PCBA and the second end of the power source, and the first portion of the uniaxially electrically conductive adhesive and the second portion of the uniaxially electrically conductive adhesive are continuous.

An exemplary uniaxially electrically conductive adhesive has a first side and an opposite second side, and the first side is electrically connected to the power source and the second side is electrically connected to the flexible PCBA.

An exemplary wearable medical device further includes a rigid PCBA, wherein the uniaxially electrically conductive adhesive electrically connects the flexible PCBA and the rigid PCBA, wherein the flexible PCBA has a first side and an opposite second side, and wherein the first side is electrically connected to the power source and to the rigid PCBA.

Another exemplary wearable medical device further includes a rigid PCBA, wherein the power source defines a longitudinal axis perpendicular to an end of the power source, the rigid PCBA is distanced from an end of the power source in a lateral direction perpendicular to the longitudinal axis, and the uniaxially electrically conductive adhesive electrically connects the flexible PCBA and the rigid PCBA.

Another exemplary wearable medical device further includes a rigid PCBA, wherein the uniaxially electrically conductive adhesive electrically connects the flexible PCBA and the rigid PCBA, wherein the flexible PCBA has a first side and an opposite second side, and wherein the first side is electrically connected to the power source and the second side is electrically connected to the rigid PCBA.

Another exemplary wearable medical device further includes a rigid PCBA, wherein: the power source defines a longitudinal axis perpendicular to an end of the power source; the rigid PCBA is distanced from the end of the power source in a longitudinal direction; and the uniaxially electrically conductive adhesive electrically connects the flexible PCBA and the rigid PCBA.

In an exemplary embodiment, the wearable medical device of claim 1 further includes a glucose sensor, wherein the uniaxially electrically conductive adhesive electrically connects the glucose sensor and the flexible PCBA.

In one aspect, the present disclosure provides a wearable medical device including a flexible printed circuit board assembly (PCBA), a battery cell having a first terminal and a second terminal, a sensor, and a uniaxially electrically conductive adhesive electrically connected to the flexible PCBA, to the first terminal and the second terminal of the battery cell, and to the sensor.

In an exemplary embodiment of the wearable medical device, the flexible PCBA is folded around the battery cell and has a first surface that is electrically connected to the first terminal and to the second terminal.

An exemplary wearable medical device includes a second PCBA, wherein the uniaxially electrically conductive adhesive is electrically connected to the second PCBA.

In an exemplary wearable medical device, a first portion of the uniaxially electrically conductive adhesive electrically connects the flexible PCBA to the first terminal, a second portion of the uniaxially electrically conductive adhesive electrically connects the flexible PCBA to the second terminal, and a third portion of the uniaxially electrically conductive adhesive electrically connects the flexible PCBA to the second PCBA.

In an exemplary wearable medical device, a first portion of the uniaxially electrically conductive adhesive electrically connects the flexible PCBA to the first terminal, a second portion of the uniaxially electrically conductive adhesive electrically connects the flexible PCBA to the second terminal and the flexible PCBA to the sensor, and the first portion of the uniaxially electrically conductive adhesive and the second portion of the uniaxially electrically conductive adhesive are discontinuous.

In an exemplary wearable medical device, the uniaxially electrically conductive adhesive comprises a single continuous substrate, wherein the single continuous substrate electrically connects to the flexible PCBA, to the first terminal and the second terminal of the battery cell, and to the sensor.

An exemplary wearable medical device further includes a bottom housing and a top housing bonded to the bottom housing, wherein an internal volume is defined between the bottom housing and the top housing, wherein the flexible PCBA, battery cell, uniaxially electrically conductive adhesive, and at least a portion of the sensor are located in the internal volume. Further, an exemplary wearable medical device includes a second PCBA located in the internal volume, wherein the battery cell is located between the second PCBA and the bottom housing.

In an exemplary embodiment of the wearable medical device, the sensor is a glucose sensor.

In another aspect, the disclosure provides a wearable medical device including a sensor, a battery cell having a first terminal and a second terminal, a rigid printed circuit board assembly (PCBA), a single piece continuous flexible printed circuit board assembly (PCBA), and a single piece continuous uniaxially electrically conductive adhesive electrically connecting the flexible PCBA to the sensor, to the first terminal, to the second terminal, and to the rigid PCBA. In an exemplary embodiment, the uniaxially electrically conductive adhesive has a first side and a second side, wherein the first side is electrically connected to the sensor, to the first terminal, to the second terminal, and to the rigid PCBA, and wherein the second side is electrically connected to the flexible PCBA. Further, in an exemplary embodiment, the sensor is a glucose sensor.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a perspective view of a low-profile wearable medical device in accordance with embodiments herein;

FIG. 2 is a cross-sectional schematic view of the low-profile wearable medical device of FIG. 1, illustrating internal components in accordance with embodiments herein;

FIG. 3 is an overhead schematic view of the low-profile wearable medical device of FIG. 2;

FIGS. 4-6 are overhead schematic views of alternative embodiments of the low-profile wearable medical device of FIG. 3;

FIG. 7 is a perspective view of a flexible PCBA for use in wearable medical device in accordance with embodiments herein;

FIG. 8 is a perspective view of a flexible PCBA and conductive adhesive for use in wearable medical device in accordance with embodiments herein; and

FIGS. 9-12 are a cross-sectional schematic views of further exemplary embodiments of low-profile wearable medical devices.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

In addition, certain terminology may also be used in the following description for the purpose of reference only, and thus are not intended to be limiting. For example, terms such as “top”, “bottom”, “upper”, “lower”, “above”, and “below” refer to directions in the drawings to which reference is made. Terms such as “front”, “back”, “rear”, and “side”, describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second”, and other such numerical terms referring to structures do not imply a sequence or order unless clearly indicated by the context.

It should be understood that various aspects disclosed herein may be combined in different combinations than the combinations specifically presented in the description and accompanying drawings.

As described herein, embodiments are provided for reducing the height and/or footprint area of wearable medical devices as compared to commercially available devices. Certain embodiments herein provide electrical connections between internal components via uniaxially electrically conductive adhesive. A “uniaxially electrically conductive” material is electrically conductive in a uniaxial direction and non-conductive in other directions. Colloquially, such material may be called z-axis electrically conductive material, as the material is conductive in the direction of the z-axis and nonconductive in the direction of the x-axis and y-axis. Typically, the z-axis direction refers to the direction of material thickness, the x-axis direction refers to the direction of material width, and the y-axis direction refers to the direction of the material length.

An example of a uniaxially electrically conductive adhesive includes conductive particles of a given size sufficiently large to span a predetermined gap between an electrical component and the conductive path. In other words, the particles are spaced a distance greater than the gap such that when pressure is applied between the electrical component and the conductive path, the particles make contact therebetween but still remained sufficiently spaced parallel to the gap to establish only uniaxial conduction. Another type of uniaxially electrically conductive adhesive relies on a concentration of conductive particles in a nonconductive resin whereby upon the application of pressure between the electrical component and the conductive path, the particles are moved together in the z-axis direction, with the resin between the electrical component and the conductive path being “squeezed out” in the x- and y-directions so that electrical contact is made between the electrical component and the conductive path through the pressure concentrated conductive particles in the adhesive. Other suitable types of uniaxially electrically conductive adhesives may be used in embodiments herein.

Certain embodiments herein provide for arranging internal components in a stack to reduce the footprint area of a wearable medical device. For example, a system-on-a-chip (SoC) or other integrated circuit device may be positioned over a power source, such as a battery cell or cells.

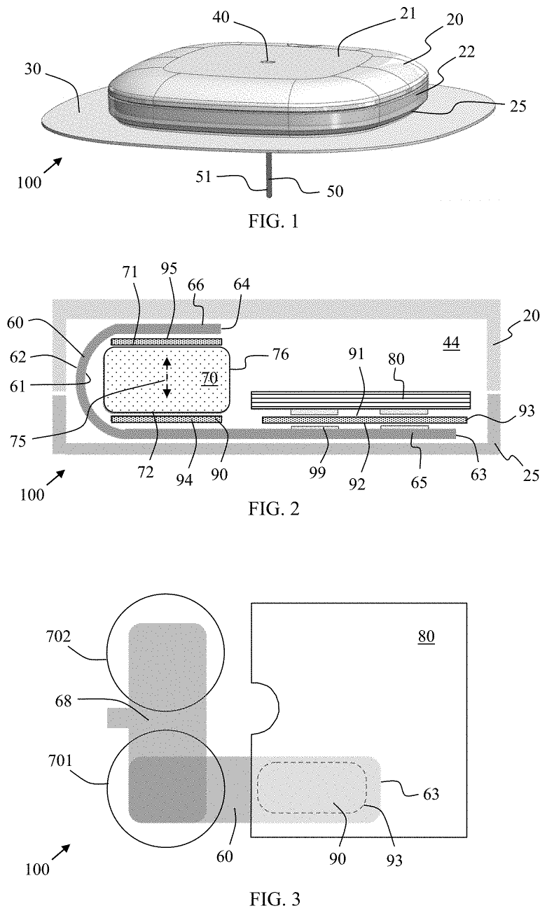

Referring to FIG. 1, a perspective view of an exemplary wearable medical device 100 is provided. While wearable medical device 100 may be used for any desired medical purpose, in an exemplary embodiment, the wearable medical device is a continuous glucose monitor (CGM) device. As shown in FIG. 1, device 100 includes a top housing 20. An exemplary top housing 20 has a horizontally-extending central portion 21 surrounding by a vertical sidewall portion 22. In an exemplary embodiment, the horizontal central and vertical sidewall portions 21 and 22 are integral with one another.

Also, the wearable medical device 100 includes a bottom housing 25. As shown, the vertical sidewall portion 22 of the top housing 20 contacts and is sealed to the bottom housing 25. As is further shown, the wearable medical device 100 may include an adhesive patch or adhesive layer 30.

In an exemplary embodiment, the top housing 20 and bottom housing 25 each include an opening 40. The opening 40 may allow a needle or probe to position a distal end of a sensor described below at a desired location for use, such as in or under the wearer's skin. The opening 40 is sealed, such as by a tubular wall. With the top housing 20 connected to the bottom housing 25 and the opening 40 sealed, an internal volume is defined and encapsulated between the top housing 20 and the bottom housing 25. As a result, internal components such as electronics are protected. In other words, the encapsulated internal volume is waterproof under normal conditions, i.e., typical environmental pressures and temperatures, so that components located within the internal volume are protected during use. As shown in FIG. 1, the wearable medical device 100 includes a sensor 50 or sensor assembly. A distal portion 51 or external portion 51 of the sensor 50 is located within the opening 40 and extends out of the device 100. A proximal portion or internal portion (not shown in FIG. 1) of the sensor 50 is located in the internal volume of the device 100. For user of the device 100, a probe may be inserted through the opening 40 to position the distal portion 51 of the sensor 50 under a user's skin. While not shown in the Figures, o-rings may be located around the opening 40 at the interface with the sensor 50 to further ensure that the internal volume is completely sealed.

Embodiments of sensors 50 provided herein use biological elements to convert a chemical analyte in a matrix into a detectable signal. In certain embodiments, a sensor 50 of the type presented here is designed and configured for subcutaneous operation in the body of a patient. An exemplary sensor 50 is a glucose sensor. The sensor 50 includes electrodes that are electrically coupled to a suitably configured electronics module that applies the necessary excitation voltages and monitors the corresponding electrical responses (e.g., electrical current, impedance, or the like) that are indicative of physiological characteristics of the body of the patient. For the embodiment described here, the sensor 50 may include a working electrode, reference electrode and counter electrode. An exemplary working electrode has includes a platinum layer, an analyte sensing layer over the platinum layer and including a catalyst or reagent or enzyme, such as glucose oxidase (GOx), a protein layer over the analyte sensing layer, an adhesion promoting layer over the protein layer, and an overlying selective permeable membrane. The working electrode may work according to the following chemical reactions:

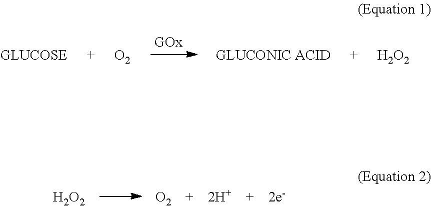

The glucose oxidase (GOx) is provided in the sensor 50 and is encapsulated by a semipermeable membrane adjacent the working electrode. The semipermeable membrane allows for selective transport of glucose and oxygen to provide contact with the glucose oxidase. The glucose oxidase catalyzes the reaction between glucose and oxygen to yield gluconic acid and hydrogen peroxide (Equation 1). The H₂O₂ then contacts the working electrode and reacts electrochemically as shown in Equation 2 under electrocatalysis by the working electrode. The resulting current can be measured by a potentiostat. These reactions, which occur in a variety of oxidoreductases known in the art, are used in a number of sensor designs.

When the sensor electrodes are placed at a subcutaneous location at a selected site in the body of a user, the sensor electrodes are exposed to the user's bodily fluids such that they can react in a detectable manner to the physiological characteristic of interest, e.g., blood glucose level. In certain embodiments, the sensor electrodes may include one or more working electrodes, counter electrodes, and reference electrodes. For the embodiments described here, the sensor electrodes employ thin film electrochemical sensor technology of the type used for monitoring blood glucose levels in the body. Further description of flexible thin film sensors of this general type are found in U.S. Pat. No. 5,391,250, entitled METHOD OF FABRICATING THIN FILM SENSORS, which is herein incorporated by reference. In other embodiments, different types of implantable sensor technology, such as chemical based, optical based, or the like, may be used.

FIG. 2 is a cross-section schematic view of the exemplary wearable medical device 100 of FIG. 1 (with certain parts eliminated for clarity). FIG. 3 is an overhead schematic view of the internal components of FIG. 2, i.e., without top housing 20 and bottom housing 25. As shown in FIG. 2, the top housing 20 and the bottom housing 25 encapsulate an internal volume 44. As shown, the illustrated device 100 includes a flexible printed circuit board assembly (PCBA) 60. As further shown, the flexible PCBA 60 has a first side 61 and an opposite second side 62 and extends from a first end 63 to a second end 64. In an exemplary embodiment, and as shown in FIG. 2, the flexible PCBA 60 is folded over itself in a C-shape, with a proximal portion 65 adjacent first end 63 and a distal portion 66 adjacent second end 64.

An exemplary flexible PCBA 60 is formed from a layer or layers of polyimide, polyethylene terephthalate (PET), or other suitable dielectric material. Conductive features, such as traces, vias, and the like are located on and/or under the first side 61 of the flexible PCBA 60, as is common for integrated circuit fabrication. The conductive features may be deposited and/or etched and may form integrated circuit components such as transistors, diodes, resistors, capacitors, inductors, and the like. Exemplary conductive features are formed as integrated circuits according to conventional fabrication processing, such that the flexible PCBA 60 may include a plurality of dielectric sublayers, and conductive layers formed therein and overlying the first side 61. In an exemplary embodiment, the flexible PCBA 60 may include a system-on-a-chip (SoC).

In FIGS. 2 and 3, the wearable medical device 100 further includes a power source 70 such as a battery cell 70 of FIG. 2 or cells 701 and 702 in FIG. 3. As shown, each battery cell 70 has a first end or first terminal 71 and a second end or second terminal 72. Exemplary battery cells are coin cells.

As further shown, in certain embodiments, the wearable medical device 100 may include a second PCBA 80. An exemplary PCBA 80 may be rigid or flexible. For a rigid member, the PCBA 80 may be ceramic material or fiberglass, i.e., glass-reinforced epoxy laminate material, such as for example FR4 composite material. For a flexible member, the PCBA 80 may be polyimide, PET, or other suitable material. Similar to flexible PCBA 60, exemplary flexible PCBA 80 may be formed from a layer or layers of dielectric material, and conductive features, such as traces, vias, and the like may be located on and/or under a surface of the PCBA 80, as is common for integrated circuit fabrication. The conductive features may be deposited and/or etched and may form integrated circuit components such as transistors, diodes, resistors, capacitors, inductors, and the like. Exemplary conductive features are formed as integrated circuits according to conventional fabrication processing, such that the PCBA 80 may include a plurality of dielectric sublayers, and conductive layers formed therein and overlying the surface of the PCBA 80. In an exemplary embodiment, the PCBA 80 may include a system-on-a-chip (SoC).

FIGS. 2 and 3 further illustrate that the wearable medical device 100 includes a uniaxially electrically conductive adhesive 90. In FIG. 2, the adhesive 90 is shown in electrical connection with the flexible PCBA 60, the power source 70, and the PCBA 80. As shown, the adhesive 90 has a first side 91 and a second side 92. Further, the illustrated adhesive 90 is discontinuous such that the adhesive 90 is formed by disconnected portions 93, 94 and 95. As shown, adhesive portion 93 is in electrical contact with the proximal portion 65 of the flexible PCBA 60 and with the PCBA 80, adhesive portion 94 is in electrical contact with the proximal portion 65 of the flexible PCBA 60 and with the second end 72 of the power source 70, and adhesive portion 95 is in electrical contact with the distal portion 66 of flexible PCBA 60 and with the first end 71 of the power source 70. In other embodiments, the adhesive 90 may be a single continuous substrate that is folded in a C-shape, such that the first side 91 contacts PCBA 80 and each end 71 and 72 of the power source 70 while second side 92 contacts the flexible PCBA 60.

FIG. 3 best illustrates that the flexible PCBA 60 includes a bridge portion 68 that extends between the battery cells 701 and 702. The bridge portion 68 is present in the distal portion 66 to provide electrical contact to the end 71 of each battery cell 701 and 702, and in the proximal portion 65 to provide electrical contact to the end 72 of each battery cell 701 and 702. It is note that while the adhesive 90 provides electrical contact between the flexible PCBA 60 and each end of the power source 70, the adhesive is hidden from view in that location in FIG. 3 and in other similar overhead views.

In the embodiment of FIGS. 2 and 3, the power source 70 defines a longitudinal axis 75 extending between and perpendicular to the ends 71 and 72. Further, as shown, the PCBA 80 is distanced from a side 76 of the power source 70 in a lateral direction perpendicular to the longitudinal axis 75.

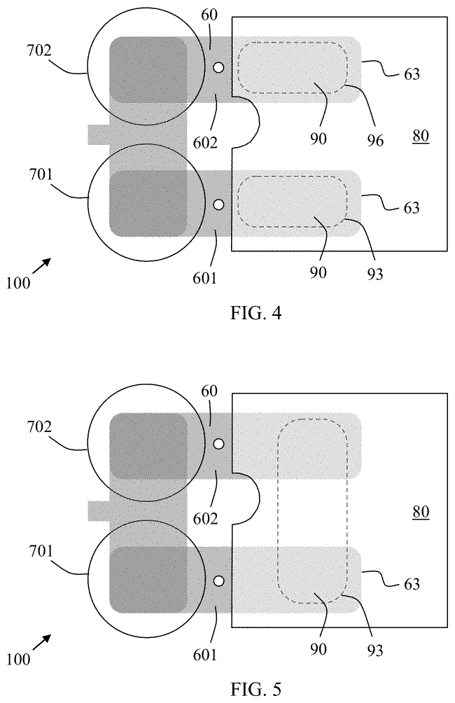

Referring now to FIG. 4, an overhead schematic view of an embodiment similar to that of FIGS. 2 and 3 is provided. As shown, the embodiment of FIG. 4 has an additional structure for electrical connection between the PCBA 80 and the power source 70 formed by battery cells 701 and 702. Specifically, in FIG. 4 (and in FIGS. 2-3) the flexible PCBA 60 includes a first leg 601 extending from battery cell 701 to the PCBA 80. In FIG. 4, the flexible PCBA 60 includes a second leg 602 extending from battery cell 702 to the PCBA 80. Further, an additional adhesive portion 96, parallel to adhesive portion 93, provide electrical contact between leg 602 and the PCBA 80.

FIG. 5 provides another embodiment similar to that of FIG. 4. In FIG. 5, a single adhesive portion 93 extends from the first leg 601 to the second leg 602 to provide electrical contact from each respective leg 601 and 602 to the PCBA 80.

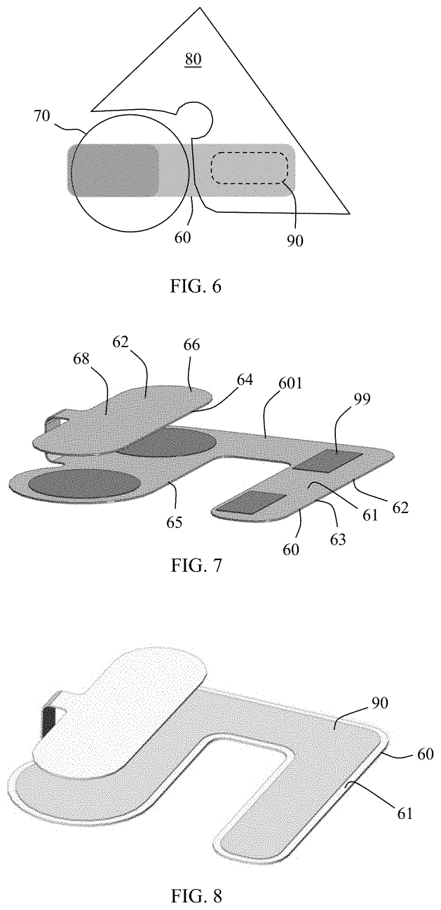

FIG. 6 is an overhead schematic view of another embodiment. In FIG. 6, the power source 70 includes a single battery cell. With an elimination of a need for footprint area for a second battery cell, the layout of the device components may be optimized to minimize the device footprint. As shown in FIG. 6, the power source 70 and PCBA 80 are arranged in a triangular layout. Again, the flexible PCBA 60 extends around and in electrical contact with each end of the power source 70 and extends to contact with PCBA 80. As in embodiments above, the adhesive 90 provides electrical contact between the flexible PCBA 60 and each end of the power source 70 (hidden from view), and between the flexible PCBA 60 and the PCBA 80.

Referring back to FIG. 2, it is noted that the device 100 may include conductive contact pads 99 at interfaces between the adhesive 90 and other components, such as between the adhesive 90 and the flexible PCBA 60. In FIG. 7, a perspective view of the flexible PCBA 60 is provided. As shown, conductive pads 99 are provided for contact to battery cells of a power source and for contact to two regions of PCBA 80, such as power (V+) and ground.

As previously described, the flexible PCBA 60 in FIG. 7 has a first side 61 and an opposite second side 62 and extends from a first end 63 to a second end 64. The exemplary flexible PCBA 60 is folded over itself in a C-shape, with the first side 61 of a proximal portion 65 facing upward and the first side 61 of a distal portion 66 facing downward. In the embodiment of FIG. 7, a single leg 601 extends from the area for contact to the power source area to the area for contact to the PCBA. Also, a bridge portion 68 in the distal portion 66 extends between areas for contact with two battery cells.

FIG. 8 provides a perspective view of the flexible PCBA 60 of FIG. 7, with an adhesive 90 located on the side 61 as a single, non-segmented, continuous member. Because the uniaxially electrically conductive adhesive 90 does not conduct electricity in the x- or y-direction, contacts formed through the adhesive 90 do not need to be physically separated from one another by gaps in the adhesive 90.

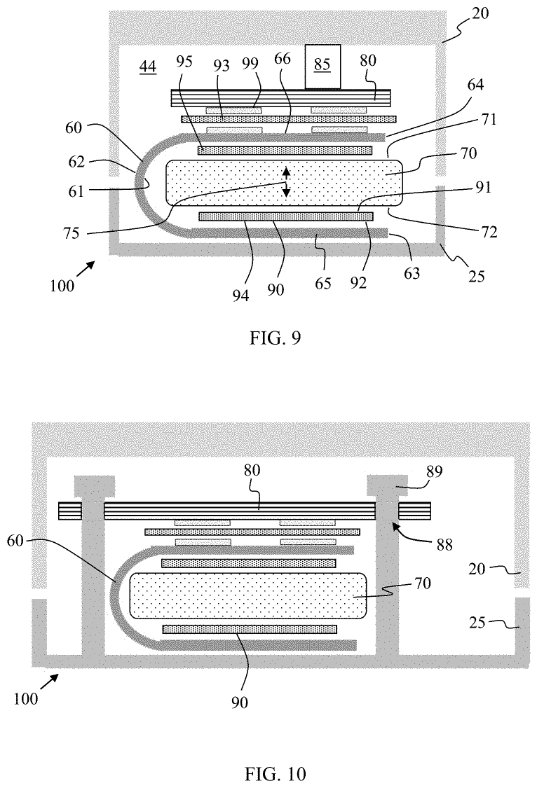

FIG. 9 is a cross-section schematic view, similar to that of FIG. 2, of another embodiment of a wearable medical device 100. In FIG. 9, the power source 70 defines a longitudinal axis 75 extending between and perpendicular to the ends 71 and 72. Further, as shown, the PCBA 80 is distanced from end 71 of the power source 70 in the longitudinal or vertical direction coincident with or parallel to the longitudinal axis 75. In the embodiment of FIG. 9, the first side 61 of the flexible PCBA 60 is electrically connected to each end 71 and 72 of the power source 70 and the second side 62 of the flexible PCBA 60 is electrically connected to the PCBA 80.

As further shown in FIG. 9, device 100 may include an expandable component 85 to exert a force onto the PCBA 80 to promote proper electrical connections through the adhesive 90. In an exemplary embodiment, the expandable component 85 is elastomeric.

FIG. 10 is a cross-section schematic view, similar to that of FIG. 9, of another embodiment for promoting proper electrical connections through the adhesive 90. Specifically, in the embodiment of FIG. 10, PCBA 80 is formed with apertures 88. Further, the device 100 includes structural posts 89 that pass through the apertures 88 of the PCBA 80 and are connected to bottom housing 25. As a result, a downward force may be applied to the PCBA 80 to promote proper electrical connections through the adhesive 90.

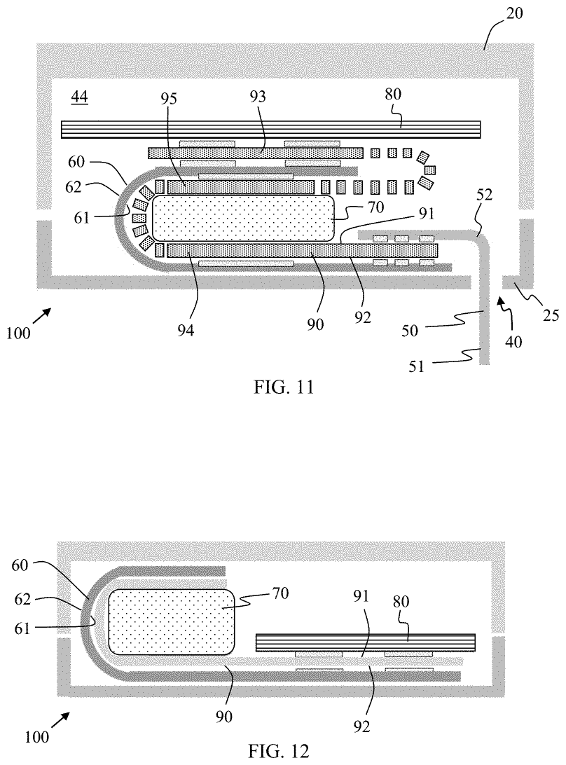

Referring now to FIG. 11, an electrical connection to the sensor 50 is shown. In FIG. 11, the proximal portion 52 of the sensor 50 is shown in the internal volume 44 of the device 100. As shown, the adhesive 90 electrically connects the sensor 50 to the flexible PCBA 60. Specifically, the sensor 50 is electrically contacted to side 91 of the adhesive 90 and side 92 of the adhesive 90 is electrically contacted to the side 61 of the flexible PCBA 60.

Also, in FIG. 11, the adhesive 90 is illustrated in dashed segments between and interconnecting the three portions 93, 94, and 95 to indicate that the adhesive 90 may be formed as a single continuous web or substrate rather than as disconnected segments. In such embodiments, the side 91 is electrically contacted to the sensor 50, to each end of the power source 70, and to the PCBA 80 while the side 92 is electrically contacted to the flexible PCBA 60.

FIG. 12 illustrates an embodiment similar to that of FIG. 2, but with an adhesive 90 in the form of a single continuous web or substrate rather than as disconnected segments. In such embodiments, the side 91 is electrically contacted to the PCBA 80 and to each end of the power source 70, while the side 92 is electrically contacted to the flexible PCBA 60.

As described herein, a wearable medical device 100 is provided with a reduced height and/or reduced footprint by providing electrical connections between internal components through the use of conductive adhesive. Further, embodiments herein provide optimal arrangements of internal components for reduced device size.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A wearable medical device comprising:
a flexible printed circuit board assembly (PCBA);
a power source; and
a uniaxially electrically conductive adhesive electrically connecting the flexible PCBA and the power source.

2. The wearable medical device of claim 1 wherein:
the power source has a first end and an opposite second end;
the flexible PCBA has a distal portion located adjacent to and electrically connected to the first end of the power source; and
the flexible PCBA has a proximal portion located adjacent to and electrically connected to the second end of the power source.

3. The wearable medical device of claim 2 wherein:
a first portion of the uniaxially electrically conductive adhesive electrically connects the distal portion of the flexible PCBA and the first end of the power source;
a second portion of the uniaxially electrically conductive adhesive electrically connects the proximal portion of the flexible PCBA and the second end of the power source; and
the first portion of the uniaxially electrically conductive adhesive and the second portion of the uniaxially electrically conductive adhesive are discontinuous.

4. The wearable medical device of claim 2 wherein:
a first portion of the uniaxially electrically conductive adhesive electrically connects the distal portion of the flexible PCBA and the first end of the power source;
a second portion of the uniaxially electrically conductive adhesive electrically connects the proximal portion of the flexible PCBA and the second end of the power source; and
the first portion of the uniaxially electrically conductive adhesive and the second portion of the uniaxially electrically conductive adhesive are continuous.

5. The wearable medical device of claim 1 wherein the uniaxially electrically conductive adhesive has a first side and an opposite second side, and wherein the first side is electrically connected to the power source and the second side is electrically connected to the flexible PCBA.

6. The wearable medical device of claim 1 further comprising a rigid PCBA, wherein the uniaxially electrically conductive adhesive electrically connects the flexible PCBA and the rigid PCBA, wherein the flexible PCBA has a first side and an opposite second side, and wherein the first side is electrically connected to the power source and to the rigid PCBA.

7. The wearable medical device of claim 1 further comprising a rigid PCBA, wherein
the power source defines a longitudinal axis perpendicular to an end of the power source;
the rigid PCBA is distanced from an end of the power source in a lateral direction perpendicular to the longitudinal axis; and
the uniaxially electrically conductive adhesive electrically connects the flexible PCBA and the rigid PCBA.

8. The wearable medical device of claim 1 further comprising a rigid PCBA, wherein the uniaxially electrically conductive adhesive electrically connects the flexible PCBA and the rigid PCBA, wherein the flexible PCBA has a first side and an opposite second side, and wherein the first side is electrically connected to the power source and the second side is electrically connected to the rigid PCBA.

9. The wearable medical device of claim 1 further comprising a rigid PCBA, wherein:
the power source defines a longitudinal axis perpendicular to an end of the power source;
the rigid PCBA is distanced from the end of the power source in a longitudinal direction; and
the uniaxially electrically conductive adhesive electrically connects the flexible PCBA and the rigid PCBA.

10. The wearable medical device of claim 1 further comprising a glucose sensor, wherein the uniaxially electrically conductive adhesive electrically connects the sensor and the flexible PCBA.

11. A wearable medical device comprising:
a flexible printed circuit board assembly (PCBA);
a battery cell having a first terminal and a second terminal;
a sensor; and
a uniaxially electrically conductive adhesive electrically connected to the flexible PCBA, to the first terminal and the second terminal of the battery cell, and to the sensor.

12. The wearable medical device of claim 11, wherein the flexible PCBA is folded around the battery cell and has a first surface that is electrically connected to the first terminal and to the second terminal.

13. The wearable medical device of claim 11 further comprising a second PCBA, wherein the uniaxially electrically conductive adhesive is electrically connected to the second PCBA.

14. The wearable medical device of claim 11 wherein:
a first portion of the uniaxially electrically conductive adhesive electrically connects the flexible PCBA to the first terminal;
a second portion of the uniaxially electrically conductive adhesive electrically connects the flexible PCBA to the second terminal; and
a third portion of the uniaxially electrically conductive adhesive electrically connects the flexible PCBA to the second PCBA.

15. The wearable medical device of claim 11 wherein:
a first portion of the uniaxially electrically conductive adhesive electrically connects the flexible PCBA to the first terminal;
a second portion of the uniaxially electrically conductive adhesive electrically connects the flexible PCBA to the second terminal and the flexible PCBA to the sensor; and
the first portion of the uniaxially electrically conductive adhesive and the second portion of the uniaxially electrically conductive adhesive are discontinuous.

16. The wearable medical device of claim 11 wherein the uniaxially electrically conductive adhesive comprises a single continuous substrate, wherein the single continuous substrate electrically connects to the flexible PCBA, to the first terminal and the second terminal of the battery cell, and to the sensor.

17. The wearable medical device of claim 11 further comprising:
a bottom housing; and
a top housing bonded to the bottom housing, wherein an internal volume is defined between the bottom housing and the top housing, wherein the flexible PCBA, battery cell, uniaxially electrically conductive adhesive, and at least a portion of the sensor are located in the internal volume.

18. The wearable medical device of claim 17 further comprising a second PCBA located in the internal volume, wherein the battery cell is located between the second PCBA and the bottom housing.

19. The wearable medical device of claim 11 wherein the sensor is a glucose sensor.

20. A wearable medical device comprising:
a sensor;
a battery cell having a first terminal and a second terminal;
a rigid printed circuit board assembly (PCBA);
a single piece continuous flexible printed circuit board assembly (PCBA); and
a single piece continuous uniaxially electrically conductive adhesive electrically connecting the flexible PCBA to the sensor, to the first terminal, to the second terminal, and to the rigid PCBA.

21. The wearable medical device of claim 20 wherein the uniaxially electrically conductive adhesive has a first side and a second side, wherein the first side is electrically connected to the sensor, to the first terminal, to the second terminal, and to the rigid PCBA, and wherein the second side is electrically connected to the flexible PCBA.

22. The wearable medical device of claim 20 wherein the sensor is a glucose sensor.
